(19) United States
(12) Patent Application Publication
(10) Pub. No.: US 2010/0184711 A1
(43) Pub. Date: Jul. 22, 2010

(54) Alpha-GLYCOSYL THIOLS AND alpha-S-LINKED GLYCOLIPIDS

(75) Inventors: Xiangming Zhu, Dublin (IE); Ravindra T. Dere, Dublin (IE)

Correspondence Address: DAVID S. RESNICK, NIXON PEABODY LLP, 100 SUMMER STREET, BOSTON, MA, 02110-2131, US

(73) Assignee: UNIVERSITY COLLEGE DUBLIN, NATIONAL UNIVERSITY OF IRELAND, DUBLIN, Dublin (IE)

(21) Appl. No.: 12/644,281

(22) Filed: Dec. 22, 2009

Related U.S. Application Data
(60) Provisional application No. 61/139,759, filed on Dec. 22, 2008.

(51) Int. Cl. A61K 31/7028; C07H 15/14; A61P 35/04; A61P 37/00; A61P 31/12

(52) U.S. Cl. 514/24; 536/17.6

(57) ABSTRACT

The present invention relates to stereoselective methods for the preparation of α-glycosyl thiols and α-S-linked glycosylceramides.

### CROSS REFERENCE TO RELATED APPLICATIONS

This application claims the benefit under 35 U.S.C. §119(e) of U.S. Provisional Application No. 61/139,759 filed on Dec. 22, 2008, the contents of which are incorporated herein by reference in its entirety.

### INTRODUCTION

The invention relates to a method for producing a stereoselective preparation of α-glycosyl thiols. The invention also relates to a method for producing a stereoselective preparation of α-S-linked glycosylceramides.

The importance of carbohydrates and glycoconjugates in numerous biochemical processes has stimulated the development of glycomimetics as fundamental tools for biological research and as potential agents for therapeutic intervention. In this context, thioglycosides have attracted considerable attention due to their resistance to chemical and enzymatic hydrolysis and their similar solution conformation and biological activities compared to native counterparts. As a consequence, many efforts have been devoted in the past two decades to synthesize thioglycosides, including thiosaccharides and S-glycoconjugates, in order to provide valuable compounds for biological studies. For instance, an S-linked glycopeptide carrying two sugar moieties has been synthesized recently in solution phase, which mimics the peptide sequence Ala484-Ala490 in the Tamm-Horsfall protein (THP). Apparently, this catabolically stable compound is of great interest for unraveling further the biological role of THP. Also, carbohydrate epitopes of conjugate vaccines have been modified to contain S-linked residues and the resulting S-linked immunogens generated an antigen-specific immune response that even exceeded the response to the native oligosaccharides.

Currently, glycosyl thiols or their precursors, such as anomeric thioacetates, which can be S-deacetylated in situ to generate the desired glycosyl thiols, are the key building blocks for the construction of thioglycosides, although thioglycosides can also be synthesized conventionally from normal glycosyl donors and the corresponding sulfur-containing acceptors. By this nonconventional approach, a variety of thioglycosides have been synthesized as stable glycoside analogues and potential agents for therapeutic intervention. For example, S-glycopeptides have been synthesized recently by two independent groups, in which glycosyl thiols were both utilized as sugar building blocks. In addition, glycosyl thiols are also useful in the synthesis of many other carbohydrate contexts, such as C-glycoside synthesis, glycosyl sulfenamide and glycosyl sulfonamide synthesis, and glycosyl disulfide synthesis.

To a great extent, the nonconventional approach for the synthesis of thioglycosides became popular owing to the chemical stability of glycosyl thiols. Unlike sugar hemiacetals, glycosyl thiols are quite stable, and the thioglycosyl anions do not mutarotate even under basic conditions. As such, the anomeric configuration of a glycosyl thiol can be maintained during the formation of its corresponding thioglycoside products, rendering the stereoselective synthesis of α- and β-glycosyl thiols extremely important. The configurationally pure β-glycosyl thiols, such as β-glucosyl thiol and β-galactosyl thiol, could be readily obtained usually by treatment of α-glycosyl halides with thiourea followed by hydrolysis with alkali metal disulfite.

However, to our knowledge, in the literature no direct procedure for the stereoselective preparation of normal α-glycosyl thiols has been reported, although α-GlcNAc- and α-GalNAc-derived anomeric thiols could be readily prepared from the corresponding per-acetylated sugars by virtue of their neighbouring acetamide groups. Only β-glycosyl chlorides have been used occasionally to prepare α-glycosyl thiols in a multi-step procedure, nevertheless, the reproducibility of this procedure is very low due to the highly reactive β-chlorides.

### STATEMENTS OF INVENTION

According to the invention, there is provided a method for producing a stereoselective preparation of α-glycosyl thiols comprising the step reacting a corresponding anhydrosugar with a sulphur nucleophile in the presence of a Lewis acid at elevated temperature.

In this specification, the term “stereoselective preparation” as applied to α-glycosyl thiols and α-S-linked glycosylceramides, should be taken to mean that the preparation includes the α-anomer, and is essentially free of the β-anomer.

The term “corresponding anhydrosugar” should be taken to mean that anhydrosugar which corresponds to the desired glycosyl thiol. Thus, in one embodiment, where the α-glycosyl thiol is α-glucosyl thiol, the corresponding anhydrosugar is anhydroglucose. In another embodiment, where the desired α-glycosyl thiol is α-galactosyl thiol, the corresponding anhydrosugar is anhydrogalactose.

In this specification, the term “elevated temperature” typically means a temperature of greater than 30° C., suitably greater than 40° C., and ideally greater than 45° C.

In one embodiment, the corresponding anhydrosugar is a 1,6-anhydrosugar having a general formula (I)

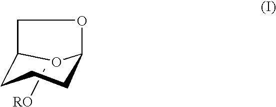

in which the or each R is independently selected from the group consisting of: Bn; All; Me; Bz; Ac; PMB; or any suitable protecting group.

In another embodiment, the corresponding anhydrosugar is a 1,5-anhydrosugar having a general formula (II)

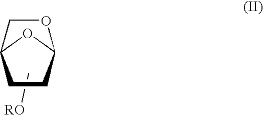

in which the or each R is independently selected from the group consisting of: Bn; All; Me; Bz; Ac; PMB; or any suitable protecting group.

Typically, the process provides a yield of α-glycosyl thiol of at least 70%, preferably at least 80%, and more preferably at least 90%. Typically, the yield means the isolated yield following chromatography.

Typically, the sulphur nucleophile is bis(trimethylsilyl)sulphide, however other sulphur nucleophiles may be used such as, for example, TrSH, MMTrSH. Suitably, the corresponding anhydrosugar is dissolved in a solvent, typically an organic solvent, containing the sulphur nucleophile. Typically, the organic solvent is dichloromethane.

Typically, the ratio of anhydrosugar to sulphur nucleophile is from 1:1 to 1:10 (mol/mol), preferably from 1:1 to 1:2 (mol/mol), most preferably about 1:1.4 (mol/mol).

Typically, the ratio of anhydrosugar to Lewis acid is from 10:10 to 10:1 (mol/mol), preferably from 10:6 to 10:2 (Mol/Mol), most preferably about 10:4 (mol/mol).

Suitably, the Lewis acid is TMSOTf.

In one embodiment, the invention provides a one-step method for producing a stereoselective preparation of α-glycosyl thiols.

The invention also relates to a stereoselective preparation of α-glycosyl thiol obtainable by the method of the invention. The invention also relates to a stereoselective preparation of an α-glycosyl thiol. The invention also relates to a stereoselective preparation of the α-glycosyl thiol selected from the group consisting of compounds 6 to 10 of Table 1. The invention also relates to the use of a stereoselective preparation of a α-glycosyl thiol of the invention in the preparation of α-S-linked glycosylceramides.

The invention also relates to a method for preparing an α-anomer of a compound of general formula (III)

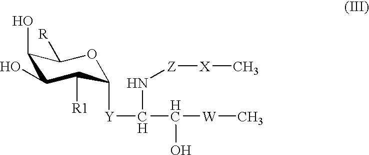

wherein
W is a saturated or unsaturated carbon chain from 9 to 15 which can contain a hydroxyl group,
X is a saturated or unsaturated carbon chain of carbon number 11 to 25 which can contain a hydroxyl group,
Y represents —S(O)₀₋₂—CH₂—,
Z represents —CO—, —SO₂—,
R represents —CH₂OH, —CO₂H, —CH₂OCH₂CO₂H, —CH₂OSO₃, and
R1 represents —OH, NH₂, —NHAc,
   the method comprising reacting an α-glycosyl thiol with a compound of general formula (IV)

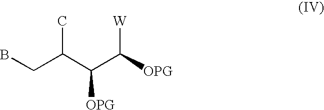

wherein
B is a leaving group,
PG represents a protecting group,
C is typically an amide or azide group,
W is a saturated or unsaturated carbon chain of carbon number 10 to 15 which may contain a hydroxyl group,
   to provide an azide intermediate, suitably reducing the azide intermediate to a corresponding amine, typically coupling an acid to the amine, and ideally deprotecting the coupling product to provide a compound of general formula (III).

Typically, the α-glycosyl thiol has a general formula (V)

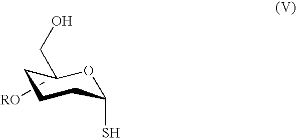

In a preferred embodiment, the PG groups in general compound (IV) are represented by an isopropylidene group. In one embodiment, the α-glycosyl thiol is selected from the group consisting of α-glucosyl thiol and α-galactosyl thiol. In one embodiment, the stereoselective preparation of α-glycosyl thiol used in the process is obtainable by the process of the invention.

The leaving B is typically selected form the group consisting of: a halogen; and a methansulfonyloxy group.

In a preferred embodiment of the invention, the stereoselective preparation of an α-glycosyl thiol with a compound of general formula (IV) is carried out under phase transfer conditions, for example in the presence of a phase transfer catalyst such as tetrabutylammonium hydrogen sulphate. Other examples of phase transfer catalysts will be apparent to those skilled in the art, including other quaternary ammonium salts and crown ethers.

Suitably, the azide intermediate is reduced by means of a Staudinger reduction, suitably using a solution of trimethylphosphine in THF.

Typically, the acid is a free acid. Preferably, the free acid is hexacosanoic acid, although any other acid, free or otherwise, may be employed.

In a preferred embodiment, deprotection of the coupling product is effected by first removing the isopropylidene group to provide a partially protected S-glycolipid, suitably by treatment with a strong acid in dioxane, and secondly by reduction of the partially-protected S-glycolipid, typically by Birch reduction.

Typically, the method of the invention is a method of preparing a stereoselective preparation of an α-anomer of the compound of general formula (III).

The invention also relates to a compound of general formula (III) obtainable by the method of the invention.

The invention also relates to a stereoselective preparation of a compound of general formula (III) obtainable by the method of the invention.

The invention also relates to an α-anomer of a compound of general formula (III), or a stereoselective preparation of an α-anomer of a compound of generally formula (III), or a pharmaceutically acceptable salt thereof,

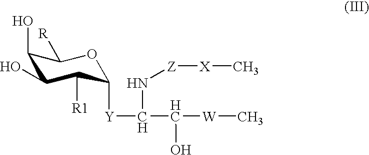

wherein
W is a saturated or unsaturated carbon chain from 9 to 15 which can contain a hydroxyl group,
X is a saturated or unsaturated carbon chain of carbon number 11 to 25 which can contain a hydroxyl group,
Y represents —S(O)₀₋₂—CH₂—,
Z represents —CO—, —SO₂—,
R represents —CH₂OH, —CO₂H, —CH₂OCH₂CO₂H, —CH₂OSO₃, and
R1 represents —OH, NH₂, —NHAc.

Preferably, R is —CH₂OH. Suitably, Y represents —SO—CH₂—. Ideally, Z represents —CO. Preferably X represents —C₂₄H₄₈. Preferably W represents C₁₃H₂₆. Typically, R₁ represents —OH.

Suitably, the compound of general formula (IV) is selected from the compounds of formulae (VI) or (VII):

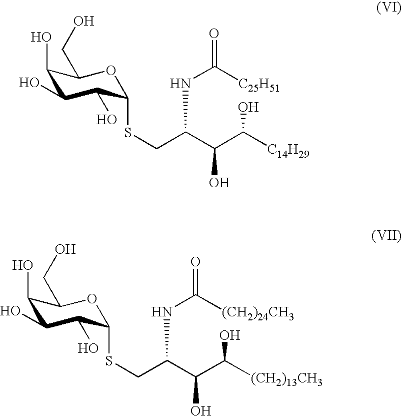

The invention also relates to an α-S-linked glycosylceramide.

The invention also relates to a stereoselective preparation of an α-S-linked glycosylceramide. Typically, the stereoselective preparation has a yield of at least 50%, preferably at least 55%, preferably at least 60%, and ideally at least 62%.

The invention also relates to a pharmaceutical composition comprising a compound of general formula (III) in combination with a suitable pharmaceutical carrier.

The invention also relates to an immunostimulating composition comprising a compound of general formula (III) in combination with a suitable pharmaceutical carrier.

The invention also relates to a method for preventing or treating cancer (or it's metastases) comprising a step of administering a therapeutically effective amount of a compound of general formula (III) to an individual.

The invention also relates to a method for preventing or treating a viral infection comprising a step of administering a therapeutically effective amount of a compound of general formula (III) to an individual.

The invention also relates to a method for preventing or treating an autoimmune disease or condition comprising a step of administering a therapeutically effective amount of a compound of general formula (III) to an individual.

In the specification, the term “individual” should be taken to means a human, however it should also include higher mammals for which the therapy of the invention is practicable.

In this specification, the term “cancer” should be taken to mean a cancer selected from the group consisting of: fibrosarcoma; myxosarcoma; liposarcoma; chondrosarcom; osteogenic sarcoma; chordoma; angiosarcoma; endotheliosarcoma; lymphangiosarcoma; lymphangioendotheliosarcoma; synovioma; mesothelioma; Ewing's tumor; leiomyosarcoma; rhabdomyosarcoma; colon carcinoma; pancreatic cancer; breast cancer; ovarian cancer; prostate cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumor; cervical cancer; uterine cancer; testicular tumor; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias. In a preferred embodiment, the cancer is selected from the group comprising: breast; cervical; prostate; and leukemias, and/or their metastases.

“Treatment or prevention” (or “treat or prevent”) as used herein includes its generally accepted meaning which encompasses prohibiting, preventing, restraining, and slowing, stopping or reversing progression, severity, of a resultant symptom. As such, the methods of this invention encompass both therapeutic and prophylactic administration.

“Effective amount” refers to the amount or dose of the compound, upon single or multiple dose administration to the patient, which provides the desired effect in the patient under diagnosis or treatment. An effective amount can be readily determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the species of mammal; its size, age, and general health; the specific disease involved; the degree of or involvement or the severity of the disease; the response of the individual patient; the particular compound administered; the mode of administration; the bioavailabilty characteristics of the preparation administered; the dose regimen selected; the use of concomitant medication; and other relevant circumstances.

In the case of cancer, the amount of the therapeutic of the invention which will be effective in the treatment or prevention of cancer will depend on the type, stage and locus of the cancer, and, in cases where the subject does not have an established cancer, will depend on various other factors including the age, sex, weight, and clinical history of the subject. The amount of therapeutic may be determined by standard clinical techniques. In addition, in vivo and/or in vitro assays may optionally be employed to help predict optimal dosage ranges. The precise dose to be employed in the formulation will also depend on the route of administration, and the seriousness of the cancer, and should be decided according to the judgment of the practitioner and each patient's circumstances. Routes of administration of a therapeutic include, but are not limited to, intramuscularly, subcutaneously or intravenously. Effective doses may be extrapolated from dose-response curves derived from in vitro or animal model test systems.

In this specification, the term “pharmaceutical composition” should be taken to mean compositions comprising a therapeutically effective amount of a compound of the invention, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound of the invention is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol and the like.

The composition, if desired, can also contain minor amounts of wetting or emulsifying agents, or pH buffering agents. These compositions can take the form of solutions, suspensions, emulsion, tablets, capsules, powders, sustained-release formulations and the like.

The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to, ease pain at the, site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In this specification, the term “immunostimulatory” should be taken to mean that the compounds of the invention modulate, and suitably increase, the immune response in a mammal to which the compound is administered. Without being bound by theory, it is believed that the compounds of the invention interact with Antigen Presenting Cells to form a complex which binds to NK cells and thereby stimulate the expression of a cytokine response in the mammal.

### BRIEF DESCRIPTION OF THE FIGURES

FIG. 1 is schematic illustration of the synthesis of α-galactosyl thiol 6 according to the method of the invention; and

FIG. 2 is a schematic illustration of the synthesis of α-S-galactosylceramide according to the method of the invention;

FIG. 3 is a schematic illustration of the synthesis of α-S-glactosylceramide according to the method of the invention; and

FIGS. 4A, 4B and 5A, 5B are graphs showing the immunostimulatory effect of the α-S-linked galactosylceramides of the invention.

### DETAILED DESCRIPTION OF THE INVENTION

### Stereoselective α-Galactosyl Thiol Preparations

α-galactosyl thiol was prepared from its corresponding 1,6-anhydrogalactose 1 (Table 1). Treatment of 1 with a small excess of bis(trimethylsilyl)sulfide in the presence of catalytic amounts of TMSOTf in which the reaction mixture was heated to 50° C., 6 was produced in very high yield as exclusively the α anomer (Table 1, entry 1). The reaction was very clean as indicated by TLC, and the anomeric configuration of the product could be readily determined by NMR spectroscopy.

A range of properly protected 1,6-anhydrosugars were then prepared following the previous procedure¹ and subjected to the above ring-opening conditions (Table 1).

TABLE 1
Synthesis of α-glycosyl thiols^{a}

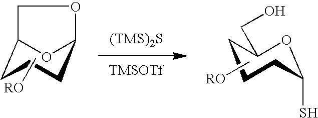

| En- | | | Yield | α/β |
|---|---|---|---|---|
| try | Substrate | Product | (%)^{b} | Ratio |
| 1 | 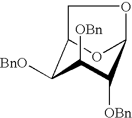 1 | 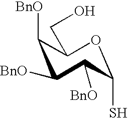 6 | 88 | α only |
| 2 | 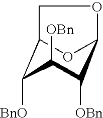 2 | 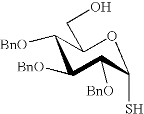 7 | 90 | α only |
| 3 | 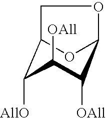 3 | 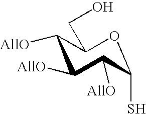 8 | 78 | α only |
| 4 | 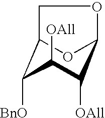 4 | 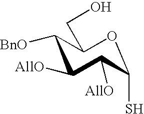 9 | 85 | α only |
| 5 | 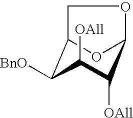 5 | 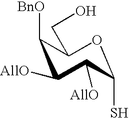 10 | 92 | α only |

^{a}All reactions were conducted under the same conditions; see text for details.
^{b}Isolated yield following chromatography.

Each substrate (1 mmol) was treated under the same conditions; it was dissolved in CH₂Cl₂ (10 mL) containing bis(trimethylsilyl)sulfide (1.4 mmol). TMSOTf (0.4 mmol) was added and the resulting mixture was heated at 50° C. until TLC indicated complete consumption of the starting material. The results, summarized in Table 1, indicate that under the above reaction conditions, 1,6-anhydrosugars can be converted effectively into α-glycosyl thiols in a stereospecific way. For instance, the benzylated levoglucosan 2 could be ring-opened with bis(trimethylsilyl)sulfide under the same reaction conditions to give the desired α-glucosyl thiol 7 in 90% yield (Table 1, entry 2). Similarly, configurationally pure thiol 8 could be produced from the corresponding allylated levoglucosan 3 in very good yield. In addition, as shown in Table 1, excellent yields and α-selectivities were also achieved for the conversion of 1,6-anhydrosugars 4 and 5 into glycosyl thiols 9 and 10, respectively. Also, in all the above reactions, no disulfide formation was detected.

The method above demonstrates a highly stereoselective method for the synthesis of α-glycosyl thiols by ring-opening of 1,6-anhydrosugars with bis(trimethylsilyl)sulfide. All the α-glycosyl thiols were isolated in high to excellent yields as exclusively the α anomer. No trace of β-isomers was produced in the reactions. Thus this one-step procedure provided a concise and efficient access to α-glycosyl thiols, which could be used to synthesize various α-S-linked glycoconjugates.

### Stereoselective α-S-Linked Glycosylceramide Preparations (Compounds VI and VII))

### Compound VI

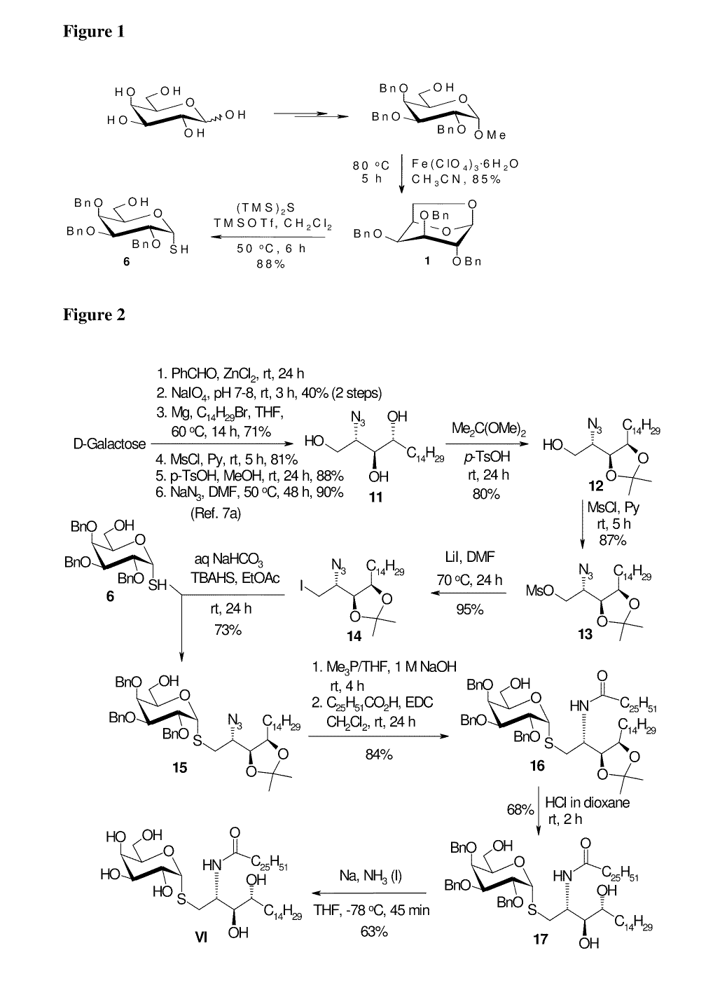

The synthesis of compound VI started with the preparation of thiol 6. 1,6-Anhydrosugar 1 was first prepared from D-galactose by literature procedures² and then selectively ring-opened with commercially available bis(trimethylsilyl)sulfide as described above to give the desired α-galactosyl thiol 6 in 88% yield (FIG. 1). The anomeric configuration of 6 was readily determined from the coupling constant: ³J_{H1-H2}=4.5 Hz, whereas analogous β-glycosyl thiols usually have ³J_{H1-H2}=7-10 Hz. This ring-opening procedure is a significant advance in glycosyl thiol chemistry because there have not been any reports on direct stereoselective preparation of α-glycosyl thiols prior to our work. Furthermore, 6 was produced exclusively as α-anomer, which made the purification very simple and straightforward.

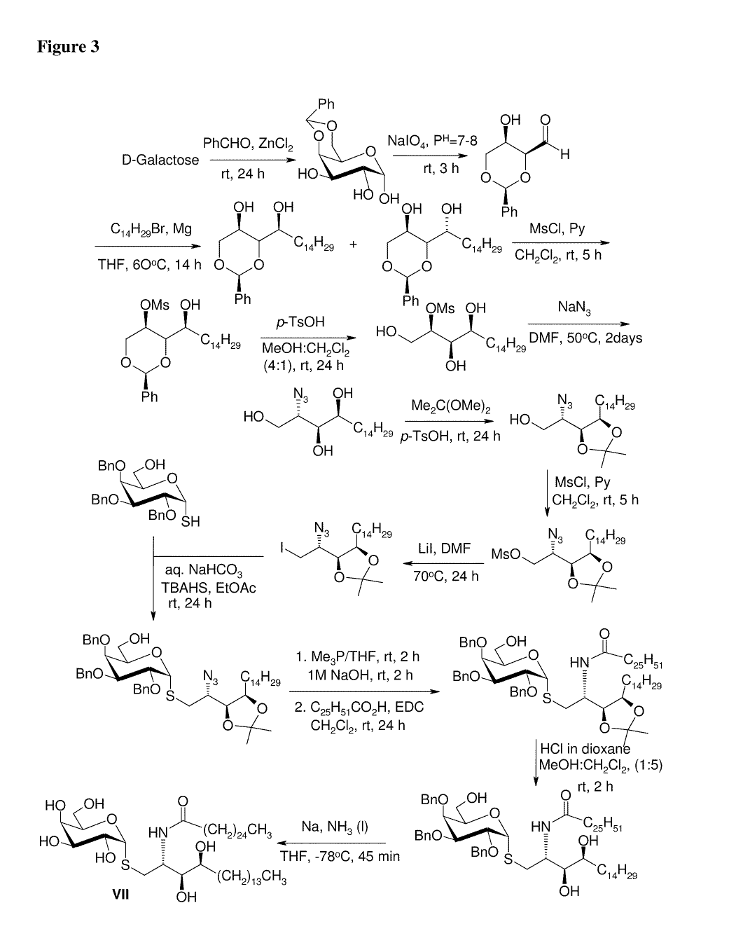

In the meantime, phytosphingosine derivative 11 (FIG. 2) was also synthesized from D-galactose following Schmidt's procedure,³ and then subjected to the normal isopropylidenation conditions (2,2-dimethoxypropane/p-TsOH) to give the intermediate 12⁴ in 80% yield. Compound 12 was subsequently mesylated with methanesulfonyl chloride in pyridine to afford a 87% yield of compound 13, which was converted into the iodide 14 in excellent yield by treatment with LiI in DMF.

With the requisite building blocks 6 and 14 in hand, coupling between thiol 6 and iodide 14 was performed in the presence of tetrabutylammonium hydrogen sulfate (TBAHS) in ethyl acetate and an aqueous solution of NaHCO₃ at pH 8.5, i.e. phase transfer conditions, as shown in FIG. 2. As expected, the desired product 15 was obtained in very good yield (73%) after SiO₂ flash chromatography, and the 6-OH group of 6 also remained inert under the conditions.

The conversion of azide 15 into the corresponding amine was effected by Staudinger reduction using 1M solution of trimethylphosphine in THF,⁵ and the amine was used directly in the acylation reaction without further purification. Coupling of the amine with hexacosanoic acid in the presence of 1-ethyl-3-(dimethylaminopropyl) carbodiimide hydrochloride (EDC) afforded the desired compound 16 in 84% overall yield. Subsequently, deprotection of 16 to convert into the target compound VI was achieved in two steps: removal of the isopropylidene protecting group was first conducted by treatment with 4M HCl in dioxane to give rise to the partially protected S-glycolipid 17 in 68% yield; 17 was then subjected to Birch reduction to furnish the α-S-galactosylceramide VI in 63% yield.

### Compound VII

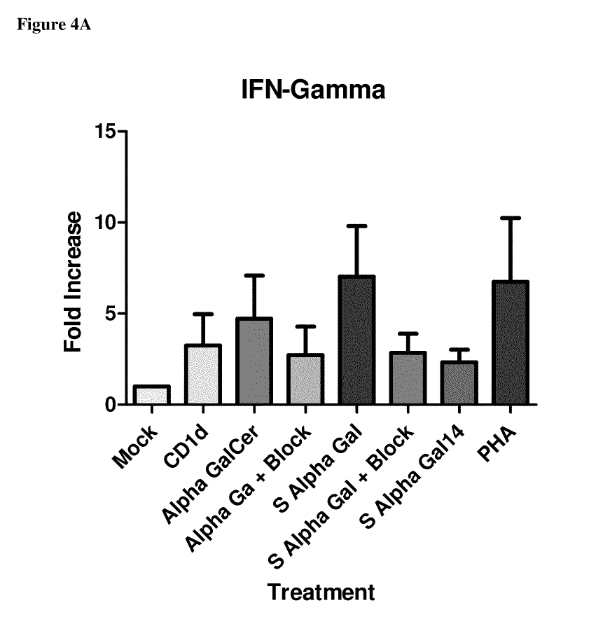

The synthesis of compound VII is illustrated in FIG. 3, and follows the same steps as the procedure described above.

In the methods described above, the phytosphingosines were prepared from D-galactose by literature procedures, however it will be appreciated that the phytosphingosine can be obtained commercially.

### NMR Data

The synthetic sample was purified by flash chromatography on silica gel (eluant: CHCl₃/MeOH 10:1), and characterized by NMR and HR-ESIMS. To liquid NH₃ (10 mL) under N₂ at −78° C. was added Na° until solution becomes blue. To the blue solution was added compound (13) (25 mg, 0.021 mmol) in THF (2 mL), and then the mixture was stirred for 45 min at −78° C. The reaction was quenched by addition of ammonium chloride until the blue colour disappeared. The NH₃ was allowed to evaporate slowly and the crude residue was purified by flash column chromatography with CHCl₃/MeOH (10:1) to afford the compound (3) (12 mg, 63%) as a white solid.

Compound VI— ¹H NMR δ (500 MHz, C₅D₅N) 8.78 (d, J=8.5 Hz, 1H), 6.10 (d, J=5.5 Hz, 1H), 5.23-5.21 (m, 1H), 5.23-5.21 (m, 3H), 4.60 (dd, J=7.5, 11.5 Hz, 1H), 4.51 (d, J=2.5 Hz, 1H), 4.41-4.36 (m, 3H), 4.26 (t, J=6.5 Hz, 1H), 3.67 (d, J=6.5 Hz, 2H), 2.55-2.51 (m, 2H), 2.32 (br s, 1H), 1.95-1.71 (m, 4H), 1.69 (br s, 1H), 1.42-1.38 (m, 2H), 1.34 (s, 27H), 1.28 (s, 41H), 0.89 (t, J=6.7 Hz, 6H); ¹³C NMR (125 MHz, C₅D₅N) δ 173.9, 89.8, 77.7, 73.6, 72.7, 72.5, 71.1, 70.2, 63.1, 53.2, 36.9, 34.0, 32.4, 32.2, 30.4, 30.2, 30.14, 30.11, 30.10, 30.04, 30.01, 29.9, 29.8, 29.72, 26.7, 26.5, 23.0, 14.3. ESI-MS m/z 874.6 [M+H]⁺. ESI-HRMS calcd for C₅₀H₉₉NO₈NaS [M+Na]⁺ 896.6989, found 896.6957.

Compound VII— ¹H NMR δ (500 MHz, C₅D₅N) 8.78 (d, J=8.5 Hz, 1H), 6.10 (d, J=5.0 Hz, 1H), 5.02-5.05 (m, 3H), 4.91-4.93 (m, 1H), 4.58 (dd, J=3.0, 13.0 Hz, 2H), 4.44 (m, 2H), 4.23-4.21 (m, 1H), 4.09 (d, J=8.5 Hz, 1H), 3.68 (t, J=7.0 Hz, 2H), 2.55 (t, J=7.5 Hz, 2H), 2.32 (br s, 1H), 1.89-1.81 (m, 4H), 1.69 (br s, 1H), 1.40-1.38 (m, 2H), 1.33 (s, 27H), 1.27 (s, 41H), 0.89 (td, J=4.0, J=6.5 Hz, 6H); ¹³C NMR (125 MHz, C₅D₅N) δ 173.9, 89.5, 77.3, 73.3, 72.4, 72.2, 70.8, 69.9, 62.8, 53.0, 36.6, 33.7, 32.1, 31.9, 30.1, 29.9, 29.83, 29.80, 29.78, 29.72, 29.69, 29.62, 29.55, 29.40, 29.38, 26.4, 26.2, 22.7, 14.0. ESI-MS m/z 874.7 [M+H]⁺. ESI-HRMS calcd for C₅₀H₉₉NO₈NaS [M+Na]⁺ 896.6989, found 896.7015.

### Biological Data

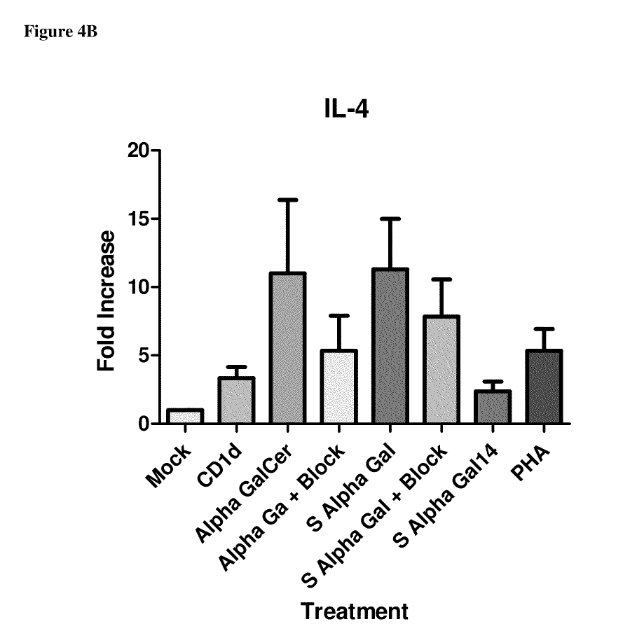

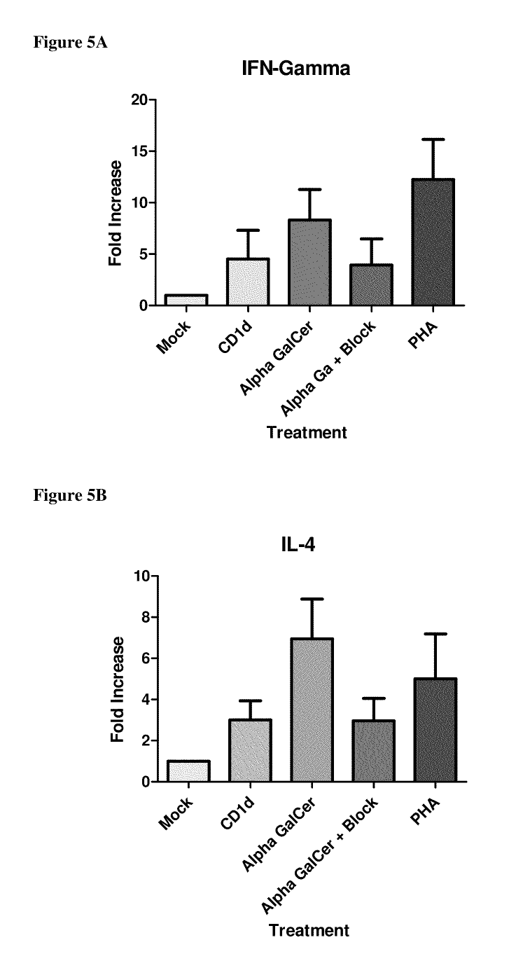

iNKT cells were treated with an α-S-linked galactosylceramide (alpha-GalCer) to determine immunostimulatory activity. FIGS. 4 and 5 show the amounts of interferon-gamma and IL-4 released (expressed as a fold-increase over the levels produced in responses to the mock-transfected cells alone) in 5 experiments. Cytokine was released when iNKT cells were co-cultured with CD1d+ cells in the absence of added glycolipid (second bar). This was enhanced for both cytokines when alpha-GalCer was added and blocked using an antibody specific for CD1d. An even greater effect was found when the S-alpha-GalCer was used and this was blocked using the antibody, indicating that CD1d is involved. The results clear show that the S-linked glycolipids are biologically active and induce both IFN-gamma and IL-4. It has also been demonstrated that the S-glycolipid, in the presence of iNKT cells, was able to stimulate the maturation of dendritic cells into antigen-presenting cells, as evidenced by phenotypic changes and the secretion of IL-12.

The invention is not limited to the embodiment hereinbefore described which may be varied in construction and detail without departing from the spirit of the invention.

### REFERENCES

1. (a) E. M. Montgomery, N. K. Richtmyer, C. S. Hudson, J. Am. Chem. Soc., 1943, 65, 3-7; (b) E. M. Montgomery, N. K. Richtmyer, C. S. Hudson, J. Am. Chem. Soc., 1943, 65, 1848-1854; (c) P. M. \acute{Å}berg, B. Ernst, Acta. Chem. Scand., 1994, 48, 228-233; (d) M. A. Zottola, R. Alonso, G. D. Vite, B. Fraser-Reid, J. Org. Chem., 1989, 54, 6123-6125.
2. \acute{Å}berg, P. M.; Ernst, B. Acta. Chem. Scand., 1994, 48, 228-233.
3. Figueroa-Pérez, S.; Schmidt, R. R. Carbohydr. Res. 2000, 328, 95-102;
4. Schmidt, R. R.; Maier, T. Carbohydr. Res. 1988, 174, 169-179; and
5. Xing, G. W.; Wu, D.; Poles, M. A.; Horowitz, A.; Tsuji, M.; Ho, D. D.; Wong, C. H. Bioorg. Med. Chem. 2005, 13, 2907-2916.

1. A method for producing a stereoselective preparation of α-glycosyl thiols comprising the step of reacting a corresponding anhydrosugar with a sulphur nucleophile in the presence of a Lewis acid at elevated temperature.

2. A method as claimed in claim 1 in which the elevated temperature is a temperature of greater than greater than 40° C.

3. A method as claimed in claim 1 in which the corresponding anhydrosugar is a 1,6-anhydrosugar having a general formula (I) 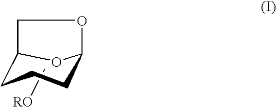
in which the or each R is independently selected from the group consisting of: Bn; All; Me; Bz; Ac; PMB; or any suitable protecting group.

4. A method as claimed in claim 1 in which the corresponding anhydrosugar is a 1,5-anhydrosugar having a general formula (II) 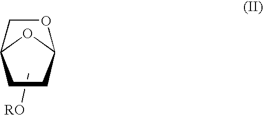
in which the or each R is independently selected from the group consisting of: Bn; All; Me; Bz; Ac; PMB; or any suitable protecting group.

5. A stereoselective preparation of α-glycosyl thiol obtainable by a method of any of claims 1 to 4.

6. A stereoselective preparation of an α-glycosyl thiol comprising an α-anomer of the α-glycosyl thiol and essentially free of the β-anomer of the α-glycosyl thiol.

7. A method for preparing an α-anomer of a compound of general formula (III) 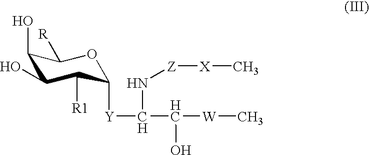
wherein
W is a saturated or unsaturated carbon chain from 9 to 15 which can contain a hydroxyl group,
X is a saturated or unsaturated carbon chain of carbon number 11 to 25 which can contain a hydroxyl group,
Y represents —S(O)₀₋₂—CH₂—,
Z represents —CO—, —SO₂—,
R represents —CH₂OH, —CO₂H, —CH₂OCH₂CO₂H, —CH₂OSO₃, and
R1 represents —OH, NH₂, —NHAc,
the method comprising reacting an α-glycosyl thiol with a compound of general formula (IV) 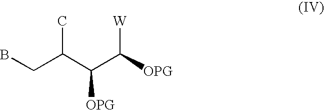
wherein
B is a leaving group,
PG represents a protecting group,
C is typically an amide or azide group,
W is a saturated or unsaturated carbon chain of carbon number 10 to 15 which may contain a hydroxyl group,
to provide an azide intermediate, suitably reducing the azide intermediate to a corresponding amine, typically coupling an acid to the amine, and ideally deprotecting the coupling product to provide a compound of general formula (III).

8. A method as claimed in claim 7 in which the α-glycosyl thiol has a general formula (V)

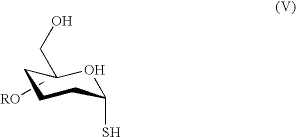

9. A compound of general formula (III), or a stereoselective preparation of an α-anomer of a compound of generally formula (III), or a pharmaceutically acceptable salt thereof, 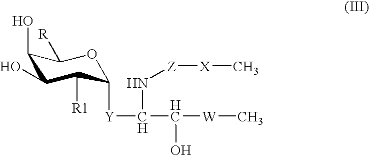
wherein
W is a saturated or unsaturated carbon chain from 9 to 15 which can contain a hydroxyl group,
X is a saturated or unsaturated carbon chain of carbon number 11 to 25 which can contain a hydroxyl group,
Y represents —S(O)₀₋₂—CH₂—,
Z represents —CO—, —SO₂—,
R represents —CH₂OH, —CO₂H, —CH₂OCH₂CO₂H, —CH₂OSO₃, and
R1 represents —OH, NH₂, —NHAc.

10. A compound according to claim 9 selected from the compounds of formulae (VI) or (VII):

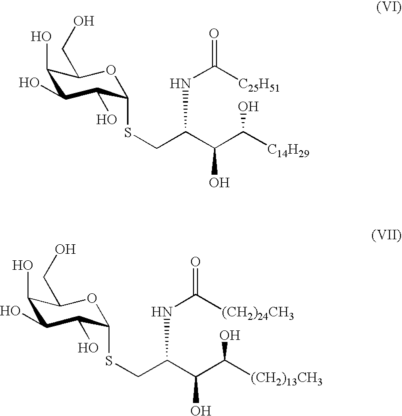

11. An α-S-linked glycosylceramide.

12. A stereoselective preparation of an α-S-linked glycosylceramide.

13. A pharmaceutical composition comprising a compound of general formula (III) in combination with a suitable pharmaceutical carrier.

14. A method for preventing or treating cancer (or it's metastases) comprising a step of administering a therapeutically effective amount of a compound of general formula (III) to an individual.

15. A method for preventing or treating a viral infection comprising a step of administering a therapeutically effective amount of a compound of general formula (III) to an individual.

16. A method for preventing or treating an autoimmune disease or condition comprising a step of administering a therapeutically effective amount of a compound of general formula (III) to an individual.
